(19) United States
(12) Patent Application Publication
(10) Pub. No.: US 2010/0234916 A1
(43) Pub. Date: Sep. 16, 2010

(54) System and method for ventricular pace timing based on isochrones

(75) Inventors: Robert Turcott, Portola Valley, CA (US); Euan Ashley, Menlo Park, CA (US)

Correspondence Address: Stanford University, 1705 EL CAMINO REAL, PALO ALTO, CA, 94306, US

(73) Assignee: The Board of Trustees of the Leland Stanford Junior University, Palo Alto, CA (US)

(21) Appl. No.: 12/722,297

(22) Filed: Mar. 11, 2010

Related U.S. Application Data
(60) Provisional application No. 61/159,247, filed on Mar. 11, 2009.

(51) Int. Cl. A61N 1/08; A61N 1/362

(52) U.S. Cl. 607/30; 607/9

(57) ABSTRACT

The present invention provides a system and method for displaying ventricular timing events and for determining optimal ventricular pace timing based on ventricular synchrony and loading conditions in order to improve the hemodynamic performance of patients.

### RELATED APPLICATION

This application claims priority and other benefits from U.S. Provisional Patent Application U.S. Ser. No. 61/159,247, filed Mar. 11, 2009, entitled “System and method for ventricular pace timing based on mechanical atrioventricular delay isochrones”. Its entire content is specifically incorporated herein by reference.

### TECHNICAL FIELD OF THE INVENTION

The present invention relates to the field of cardiac pacing and, in particular, to a method and system for displaying ventricular timing events and for determining optimal ventricular pace timing based on ventricular synchrony and loading conditions in order to improve the hemodynamic performance of patients.

### BACKGROUND

The heart is the center of the circulatory system where, in the healthy heart, electrical pulses propagate to the heart muscle tissue to induce the atrial and ventricular contractions necessary to continuously provide oxygen-rich blood to the organs of the body. Oxygen-depleted blood flows from the peripheral venous system to the right atrium (RA), from the right atrium to the right ventricle (RV) through the tricuspid valve, from the right ventricle to the pulmonary artery through the pulmonary valve, to the lungs (pulmonary circulation). Oxygen-rich blood from the lungs is then drawn from the pulmonary vein to the left atrium (LA), from the left atrium to the left ventricle (LV) through the mitral valve, and finally, from the left ventricle to the peripheral arterial system through the aortic valve (systemic circulation). The left and right atria contract at approximately the same time, pushing blood into the left and right ventricles, respectively.

The electrical signature of the contraction of the left and right atria is the P wave, which is visible on surface ECG. Shortly after the atrial contraction, the left and right ventricles contract and eject blood into the systemic and pulmonary circulation, respectively. The strength of ventricular contraction and its mechanical efficiency are influenced by the amount of ventricular preload, i.e., the amount of blood in the ventricle, and the synchrony of contraction, i.e., how spatially uniform the electrical excitation is when contraction begins. The period of ventricular relaxation and filling is referred to as ‘diastole,’ and the period of contraction is referred to as ‘systole.’ Thus, the timing between the atrial contraction and ventricular systole determines the preload of the ventricle and influences the strength of its contraction. Other factors that influence preload include the amount of valvular regurgitation and stenosis, and synchrony of ventricular contraction, as described below.

In the chronically failing heart, the electrical conduction within the heart frequently becomes abnormal; often, conduction is delayed or blocked entirely. For example, left bundle branch block is commonly seen in heart failure patients and refers to the failure of the conduction system of the left ventricle (the ‘left bundle branch’) to conduct. In this case, electrical propagation might proceed through the myocardium, i.e., the muscle tissue, which is significantly slower than propagation through the normal conduction system. As a result of slow and delayed propagation, the atrial and ventricular contractions become dyssynchronous, which results in less forceful and less efficient pumping of the heart and insufficient supplying of the organs of the body with oxygen-rich blood. In the United States, there are currently approximately 5 million patients who suffer from heart failure with approximately half a million new diagnoses per year. Cardiac resynchronization therapy (CRT) has crystallized as the only non-pharmacologic therapy for patients with conduction abnormalities and impaired systolic function; between 1990 and 2002, about 2.3 million cardiac pacemakers and 400,000 implantable cardioverter defibrillators were placed.

Cardiac resynchronization therapy by biventricular pacing is a promising therapy in patients with heart failure associated with asynchrony of left ventricular (LV) contraction to improve the conduction pattern and sequence of the heart (Cazeau et al., 2001; Abraham et al., 2002; Auricchio et al., 2002). In conventional or CRT pacemakers the beforementioned P wave is detectable by a cardiac pacemaker as electrical activity on the right atrial lead. Alternatively, the pacemaker can initiate an atrial contraction by delivering a pace pulse to the right atrial lead. After an atrial contraction is sensed or an atrial pacing pulse is delivered, the CRT device then paces both the right and left ventricles. This restores the ventricular synchrony that is lost with the conduction abnormalities of heart failure, and is in contrast to conventional pacemakers which typically will only pace or sense at one ventricular location, commonly the right ventricular apex. In biventricular pacemakers (i.e., CRT devices) the timing between the right and left ventricular paces influences the synchrony of contraction as well as the end of diastole and the onset of systole.

Ventricular loading conditions significantly influence the strength of contraction. If the myocardium is lightly loaded, i.e., the end-diastolic pressure and volume are low, then relatively little force is generated with the next contraction. As the preload is increased, i.e., greater mechanical stress is experienced by the myocardium, which is associated with increased end-diastolic pressure and volume, the strength of contraction progressively increases through the well-known Frank-Starling relationship. As the preload is increased still further the contraction strength can actually start to decrease. Thus there is an optimum degree of preload such that the strength of contraction is maximized.

Two important parameters in cardiac resynchronization therapy are (i) atrioventricular delay or “AVD”, which is the interval between atrial event (either intrinsic contraction which is sensed by pacemaker or a paced contraction which is initiated by the pacemaker with a pace pulse) and ventricular pace; and (ii) interventricular interval (VVI), which is the interval between ventricular paces.

All major pacemaker and implantable defibrillator manufacturers allow programming of pace timing by letting the clinician specify the nominal programmed electrical AVD and VVI via an external programmer. However, because the number of all possible combinations of AVD and VVI is too large to allow exhaustive testing, the AVD and the VVI are routinely optimized independently under the likely erroneous assumption that these parameters independently determine preload and dyssynchrony. In fact, mathematical modeling and emerging data indicate that the mechanical AVD and hence LV preload are influenced by both RV and LV pace timing, so that adjustment to the programmed VVI, even with a fixed programmed AVD, results in changes in LV preload.

Precise timing of ventricular contraction can profoundly improve clinical outcomes in heart failure patients. It is, therefore, necessary to find new, accurate ways to represent and determine ventricular pacing and timing events, since this will improve efficiency and accuracy of pacemaker optimization, and, so, the hemodynamic performance and clinical outcomes for the patients who seek to benefit from those pacemakers.

### SUMMARY

Embodiments of the present invention address the problem of inadequate representation of cardiac timing events and suboptimal timing of ventricular contraction in conventional cardiac resynchronization therapy (CRT) devices and features a system and method for improving atrioventricular and interventricular interval optimization based on a new representation of ventricular timing and the concept of ventricular loading isochrones. Advantageously, this approach (i) allows identification of true pace-timing optimization with improved cardiac function; (ii) reduces time required for optimization; and (iii) presents complex timing information in a much more intuitive format for the clinician, and provides so for more efficient pacing optimization, which is expected to translate into better clinical outcomes for patients using CRT devices.

The above summary is not intended to include all features and aspects of the present invention nor does it imply that the invention must include all features and aspects discussed in this summary.

### INCORPORATION BY REFERENCE

All publications, published patent applications and patents mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings are not to scale.

FIG. 1 is an example of the graphical representation of electrical events in the ta-LV, ta-RV plane. Such a graphical representation can be presented by the programmer to facilitate parameter selection and interval optimization as well as communication of intrinsic timing information. Diagonal dotted lines show VVI isochrones. Curved dotted lines show ventricular loading isochrones. Grey solid lines show the manufacturer's programmed AVD isochrones (here, programming an AVD of 120 msec and then changing the VVI from one extreme to the other will execute a trajectory in the plane that corresponds to the grey line). ‘\’ indicates intrinsic LV conduction, either by conduction through the AV node or from a pace in the contralateral ventricle. ‘/’ indicates intrinsic RV conduction, either by conduction through the AV node or from a pace in the contralateral ventricle. ‘X’ indicates intrinsic biventricular conduction.

Fine dots represent all programmable settings that are available. Coarse dots represent previously programmed settings for the particular patient. The red dot indicates current programmed values. When the cursor is moved over a particular point a window can open which shows the timing values corresponding to that point both in terms of the ta_LV, ta_RV representation and in terms of conventional interval definitions (i.e., programmed AVD and VVI). It can also show other information such as when the intervals were programmed and the basis for making that programming decision. It can also report the mechanical AVD or preload that corresponds to the particular point in the plane. In addition, the graphical representation can also show locked out parameter values, i.e., specific values of pacing intervals that are not allowed because they would conflict with the particular values that have been programmed for other adjustable parameters, such as pace refractory time and tachycardia detection parameters.

FIG. 2 illustrates the drawback of conventional pacing optimization and the benefit of programming along ventricular preload isochrones. In conventional pacemakers and programmers, the programmed AVD does not correspond to a unique preload that remains constant as the VVI is changed. In the left-hand panel, ventricular preload isochrones (dotted lines) depict the combinations of ta-LV, ta-RV pairs that yield a constant ventricular preload. In particular, they show the combinations of ta-LV and ta-RV that result in the same preload that results from programmed settings of VVI=0 and the specific programmed AVD defined by the intersection of the preload isochrone and the VVI=0 isochrone. For example, the trajectory indicted by the bold arrow corresponds to a change from simultaneous biventricular pacing with a programmed AVD of 120 msec (solid circle) to a VV interval of −80 msec (open circle) in which the ventricular preload is held constant.

In contrast, with Medtronic and St Jude Medical devices, increasing the VV interval (thick solid line) to +/−80 msec (solid triangles) while holding the programmed AV delay fixed at 120 results in an increased mechanical AV delay and an increased preload. For Boston Scientific devices, increasing the VV interval (thick dashed line) with a fixed programmed AV delay decreases the mechanical AV delay and reduces preload. These effects are summarized in the right-hand panel, in which holding the programmed AVD fixed while changing the VVI results in an increase in preload for Medtronic and St Jude devices (solid line) and a decrease in preload for Boston Scientific devices (dotted line). In contrast, programming along a fixed preload isochrone as the VV interval is changed, by definition, maintains a fixed preload as the VV interval is adjusted (dotted line).

FIG. 3 illustrates a flow chart outlining the various steps of the automatic optimization process: automatic representation of intrinsic electrical timing, possible programmable settings, previously programmed settings, current setting, VVI isochrones, programmed AVD isochrones, and preload isochrones. The sample resolution (here 10 msec) can be made larger or smaller. An “atrial event” can be sensed atrial activity or atrial pace. Two analogous plots can be generated: one for atrial sense/ventricular pace, and one for atrial pace/ventricular pace. I.e., the process can be repeated for both atrial sensed and atrial pace, yielding two ta_RV/ta_LV plots that show time of intrinsic ventricular conduction. Instead of arrival time as outlined here a similar algorithm could be used to identify time to loss of capture. This should yield similar results to arrival times. The preload isochrones can be obtained from a theoretical model, or from empirical data from a population of patients, or from empirical data from the particular patient whose pacemaker is presently being programmed. To speed the construction of the plot the intrinsic timing information can be periodically and automatically by the pacemaker so that the information is available when the patient arrives in clinic or the pacemaker data is remotely downloaded.

FIG. 4 illustrates a flow chart outlining the automatic calculation of ta_RV, ta_LV with fixed mechanical AVD, a particular form of preload isochrones. This calculation applies both to generating timing information that is used by the pacemaker, and for generating the graphical representation of mechanical AVD isochrones.

FIG. 5 illustrates the display of intrinsic conduction information for a patient with a right bundle branch block. Also shown are VVi isochrones and specific programmed intervals of AVD=120, VV=0 (solid square), AVD=120, VV=−40, ie, LV first, which maps to either the solid circle or open circle, depending on the manufacturer.

FIG. 6 illustrates the display of intrinsic conduction information for a patient with a left bundle branch block. Also shown are VVi isochrones and specific programmed intervals of AVD=120, VV=0 (solid circle), RV only pacing at a programmed AVD of 120 (upward pointing solid triangle), LV only pacing at a programmed AVD of 120 (downward pointing solid triangle), and intrinsic conduction with no ventricular pacing (solid square).

FIG. 7 illustrates various forms of preload isochrones, in which the amount of preload is equivalent to the preload that occurs with a programmed AVD of 120 and simultaneous biventricular pacing, i.e., VV=0. The dotted curve, labeled ‘a’, results when the programmed AV delay is held fixed and increases in VV interval (either positive or negative) have an overall effect of increasing preload. The solid curve, labeled ‘b’, results when there is no change in preload as the VV interval is changed and the programmed AV delay is held fixed. The dashed curve, labeled ‘c’, results when increases in VV interval (positive or negative) decrease the preload.

FIG. 8 illustrates individual examples of a family of preload isochrones that are concave up.

FIG. 9 illustrates individual examples of a family of preload isochrones that are orthogonal to the VVi isochrones. These represent a special case in which preload remains constant when the average of ta-RV, ta-LV is held fixed.

FIG. 10 illustrates individual examples of a family of preload isochrones that are concave down.

FIG. 11 illustrates individual examples of a family of preload isochrones that are piecewise linear. Here, for VVi that is small in magnitude (positive or negative), preload remains fixed when the average of ta-RV, ta-LV is constant, however, for larger VVi the preload depends only on the time to the first-paced ventricle.

### DEFINITIONS AND ABBREVIATIONS

The term “telemetric device”, as used herein, relates to a medical device that communicates by telemetry with a pacemaker or defibrillator that is implanted in a patient with a cardiac disorder.

AVD or AVd denominates atrioventricular delay.

VVI or VVi denominates interventricular interval.

LV denominates left ventricle or left ventricular.

RV denominates right ventricle or right ventricular.

[tA-LV denominates the time from atrial event to LV pace.

tA-RV denominates the time from atrial event to RV pace.

An “atrial event” can be sensed atrial activity or atrial pace.

Mechanical AVd denominates the time between atrial event and ventricular event.

Preload isochrones describe a collection of (tA-RV, tA-LV) that yield a given ventricular preload.

“Ventricular preload” is used in the generic sense to refer to any measure of ventricular loading, including but not limited to mechanical AVD, end-diastolic pressure, end-diastolic volume, myocardial stress, diastolic filling time, and mitral regurgitation.

Mechanical AVd Isochrones describe a collection of (tA-RV, tA-LV) that yield a given mechanical AVd.

VVi isochrones describe a collection of (tA-RV, tA-LV) that yield a given VVi. An isochrone is a line or curve on a plot that demarcates all points which have the same time of occurrence or value of a particular phenomenon or of a particular value of a quantity.

dP/dt denominates the time course of left ventricular pressure.

### DETAILED DESCRIPTION

It is the overall goal of the present invention to improve the efficiency and accuracy of pacemaker programming and in particular of timing optimization, as these parameters directly influence the hemodynamic performance of the patients who are in need of cardiac resynchronization therapy (CRT). A further goal is to provide a rich representation of ventricular timing information which will enable the clinician to make more informed programming decisions.

Two important programmable parameters that are associated with cardiac resynchronization therapy are (i) atrioventricular delay or “AVD”, which is the interval between atrial contraction (either intrinsic contraction and sensed by pacemaker or initiated by the pacemaker with a pace pulse) and ventricular pace; and (ii) interventricular interval (VVI), which is the interval between ventricular paces. Optimizing either atrioventricular or interventricular delay improves cardiac performance in patients with biventricular pacemakers. However, the lack of a standard method for optimization has led in many cases to suboptimal device optimization and, consequently, to a suboptimal performance of a pacemaker.

An embodiment of the present invention enables ‘one-click programming’ so that both the AVD and VVI can be changed with a single user input.

### Cardiac Function

Cardiac function is influenced by the timing of ventricular paces relative to the atrial event. “Pacing interval optimization” is the process by which the pacing timing that yields the best cardiac function is identified. A wide variety of optimization techniques have been advocated and are in use, including multiple approaches to the assessment of systolic function, diastolic function, and electrical and mechanical synchrony (Morales et al., 2006; Burri et al, 2006; Agler et al, 2007; Jansen et al., 2006; Heinroth et al., 2007; Braun et al, 2005; Tse et al, 2003; Bertini et al, 2008; van Gelder et al, 2008; Burri et al, 2005; Chung et al, 2008; Vidal et al, 2007; Turcott et al, 2008). For example, optimization of stroke volume by measurement of blood flow velocity through the aorta using Doppler echocardiography is one way to assess systolic function (Agler et al, 2007). Measurement of rate of change of LV pressure using a catheter is another way of assessing systolic function (van Gelder et al, 2008). Diastolic function can be optimized by assessing the filling pattern through the mitral valve using Doppler echocardiography (Agler et al, 2007). We use “optimization” in the generic sense to refer to any technique whereby pacing intervals are adjusted such that cardiac function is improved.

### Ventricular Preload

Factors that influence the degree of preload include the interval from atrial contraction to ventricular contraction, the interval between left and right ventricular contraction, the degree of mitral and aortic regurgitation and stenosis, the diastolic filling time, and the amount of blood that was ejected from the ventricle during the previous heart beat, which influences the residual ventricular volume at the end of systole. In addition, other factors also play a role such as regional differences in contraction timing within the ventricle, the distribution of myocardial strain, and the relative timing of ventricular stretch and contraction with the atrial and ventricular contraction, respectively. While electrical systole can be defined by the electrical activity of the ventricle, mechanical systole is defined by mechanical events, such as the closure of the mitral valve at the end of diastole or an increase in ventricular pressure or rate of change of pressure above defined thresholds (eg, 10% of the maximum value). The time to onset of mechanical systole influences the loading of the ventricle. Thus multiple factors interact in a complex way to influence the loading condition of the ventricle.

### Cardiac Timing Events

Mechanical atrioventricular delay (AVD) is the timing from an atrial event (sensed electrical activity or delivered pace) to the onset of left ventricular systole and influences ventricular loading conditions, while the nominal programmed AVD is defined by the timing of electrical events in the pacemaker, i.e. AVD onset is determined by atrial sense or pace and AVD termination is determined by ventricular pace. Other factors that influence ventricular loading include ventricular synchrony and contractility, diastolic filling time, isovolumic contraction and relaxation times, and mitral regurgitation, all of which are potentially influenced by the VVI.

The ventricular preload changes as the programmed VVI changes, even for a fixed nominal electrical AVD. Consequently, the conventional approach implemented by manufacturers and used by clinicians results in a changing mechanical AVD as the VVI is adjusted even though the nominal programmed electrical AVD may be held constant. As a result, optimizing the VVI moves the mechanical AVD away from the optimum that was previously identified during AVD optimization.

### Implantable Cardiac Stimulation Devices

Implantable cardiac stimulation devices, such as cardiac pacemakers and implantable cardioverter defibrillators, are usually configured to be used in conjunction with an external programmer that enables a physician to program the operation of an implanted device to, for example, control the specific parameters by which the pacemaker functions and by which it detects electrical rhythm disorders and responds thereto. The programmer also downloads information from the device, for example, timing information that tells when electrical activity is sensed by the various leads, history of observed arrhythmias, functional aspects of the device such as battery energy level and lead impedances, etc.

### Limitations of Conventional Implantable Cardiac Stimulation Devices and Programmers

All major pacemaker and implantable defibrillator manufacturers allow programming of pace timing by letting the clinician specify the nominal programmed electrical AVD and VVI via an external programmer. However, because the number of all possible combinations of AVD and VVI is too large to allow exhaustive testing, the AVD and the VVI are routinely optimized independently under the likely erroneous assumption that these parameters independently determine preload and dyssynchrony. In fact, mathematical modeling and emerging data indicate that the mechanical AVD and hence LV preload are influenced by both RV and LV pace timing, so that adjustment to the programmed VVI, even with a fixed programmed AVD, results in changes in LV preload. For example, with a fixed programmed AVD as the VVI is increased the time to the onset of mechanical ventricular systole is increased, which potentially extends diastolic filling time and hence preload. On the other hand, as VVI increases the synchrony of ventricular contraction may be compromised, which can result in increased isovolumic contraction and relaxation times and thus shorten the total diastolic filling time, thereby reducing preload. Still another factor is that mitral regurgitation can be exacerbated by dyssynchrony, which further decreases preload. Furthermore, while the nominal programmed electrical AVD and VVI uniquely determine the timing of the RV and LV paces relative to the atrial event, i.e., the atrial pace or sensing of intrinsic contraction, the mapping from programmed AVD and VVI to RV and LV pace timing varies from manufacturer to manufacturer.

Another problem is that the conversion from the programmed AVD and VVI to ventricular pace timing is then performed by an external, telemetric device and/or pacemaker and is not transparent to the clinician. Understanding the conversion from programmed AVD and VVI to delivered pace timing requires highly detailed knowledge of the design of devices from various manufacturers, which is challenging and cumbersome for the practicing clinician. Frequently, pacing intervals are not optimized and instead the manufacturer's default settings are used or intervals are programmed ‘empirically’, i.e., the clinician selects what he or she thinks are reasonable parameter values based on consideration of factors such as underlying cardiac disease, PR interval on the ECG, ventricular chamber size, and location of pacing leads.

When pacing interval optimization is performed, in an attempt to find the overall best pacing combination, VVI is typically kept fixed (e.g., at 0 msec) while the AVD is optimized; subsequently VVI is optimized while the AVD is held fixed {Boriani et al., 2006; Burri et al., 2006). The assumption behind this approach is that the programmed AVD controls preload and the programmed VVI independently controls synchrony. However, although it is not widely appreciated, the programmed AVD is in fact distinct from the mechanical AVD and it is the latter which determines preload, along with other effects such as degree of mitral regurgitation, end-systolic volume, isovolumic contraction and relaxation times, etc, as discussed above. Since the ventricular loading is influenced by the RV and LV pace timing, changing the VVI in an attempt to optimize synchrony while holding the programmed AVD fixed will in fact change both synchrony and mechanical AVD. Measures of cardiac function obtained with a changing VVI and fixed programmed AVD thus reflect inextricably confounded changes in synchrony and preload. Ultimately, optimizing the VVI moves the ventricular preload away from the optimum that was originally identified during AVD optimization. Conventional programmers do not represent the ventricular preload isochrones, i.e., the combinations of pacing parameters that yield a fixed degree of preload. Thus it is difficult to adjust the programmable pacing intervals such that an originally identified optimum preload is held constant and synchrony is optimized.

Other drawbacks to the conventional representation of pace timing, i.e., the programmed AVD and programmed VVI, include ambiguity about the precise timing of ventricular activation, uncertainty about intrinsic electrical events (such as intrinsic conduction to each ventricle), and cumbersome representation of pace timing and pacing protocols.

A further limitation of conventional programmers is that adjusting the programmed pacing intervals (AVD and VVI) is cumbersome, time-consuming, and hampered by limited information about the patient's intrinsic conduction properties. For example, to change both the AVD and VVI typically requires navigating through 10 different screens on the conventional programmer. No information is provided about intrinsic conduction times or conduction patterns, such as whether the patient has a bundle branch block.

Still other drawbacks to the conventional representation of pace timing include an inability to efficiently represent intrinsic timing information. For example, it is helpful for the clinician to know whether the patient's intrinsic conduction is unusually long or short. The reason for this is that CRT devices are only effective if they successfully pace both chambers of the heart on the majority of beats, thus the physician wants to program an AVD that is sufficiently short to ensure biventricular capture. It is also useful to know whether the patient has a conduction abnormality, such as left or right bundle branch block. Furthermore, it is helpful to know what the intrinsic conduction time to the contralateral ventricle is at a given AVD. All of this information is difficult to efficiently convey using the text-based approach of conventional programmers.

### Utility of the Present Invention

Precise timing of ventricular contraction can profoundly improve clinical outcomes in heart failure patients. Having recognized that the way conventional pacemakers and programmers represent pacing interval parameters is cumbersome and inefficient, and in addition leads to a deviation from the optimal atrioventricular delay and, so, to a suboptimal performance of a pacemaker, the inventors of the present invention developed a system and method for improved pacemaker timing representation and optimization by converting programmed electrical atrioventricular delay and interventricular interval into ventricular pace timing in a way that adjustments to the interventricular interval maintain a fixed ventricular preload, i.e. occur along a given preload isochrone.

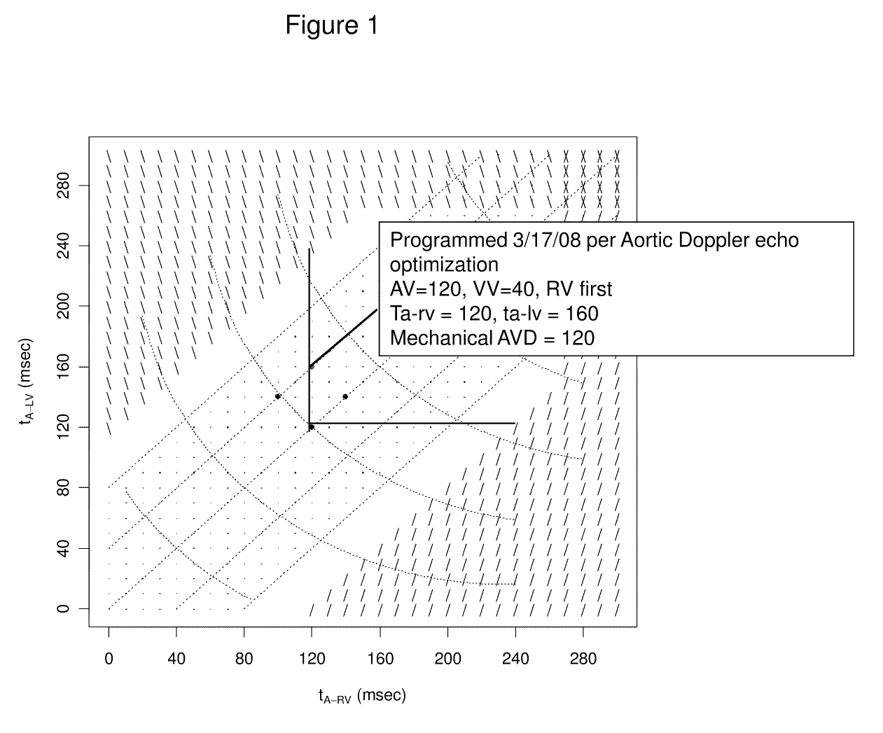

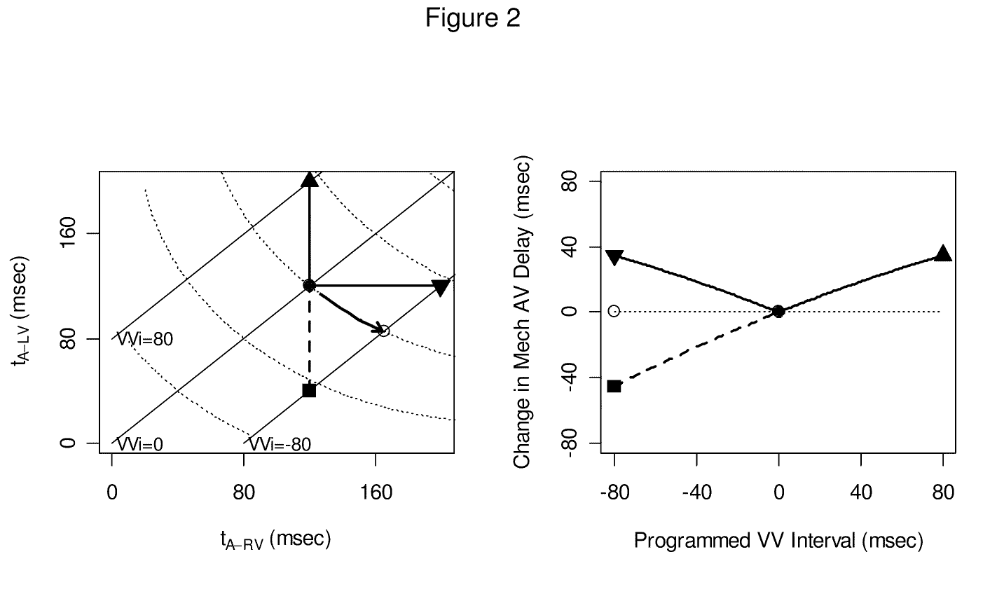

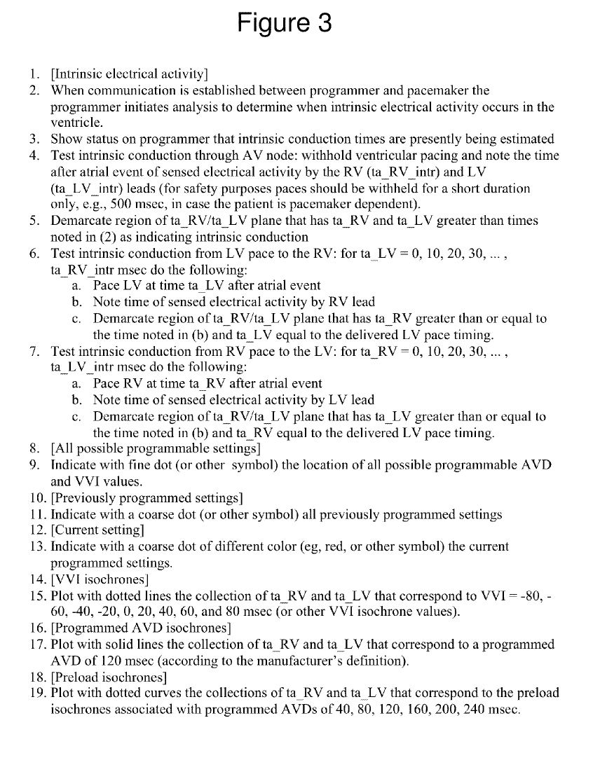

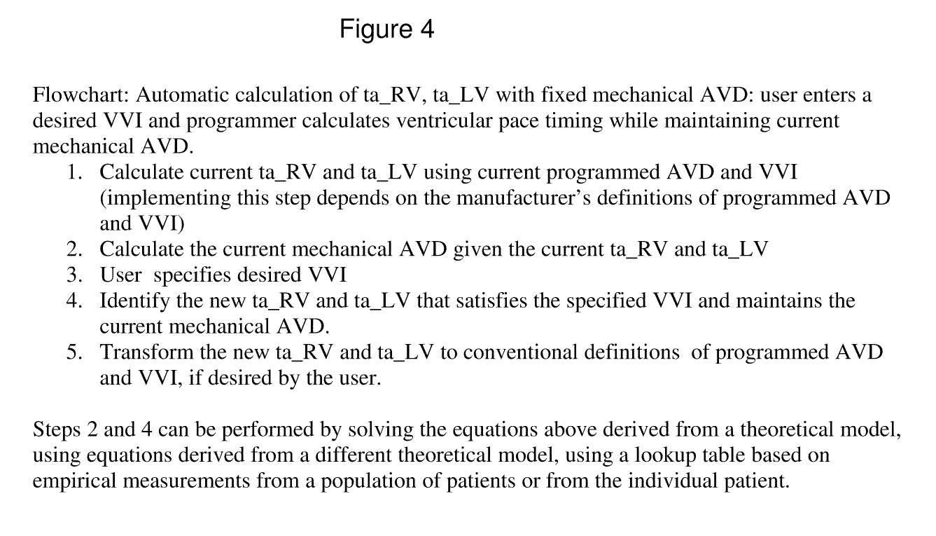

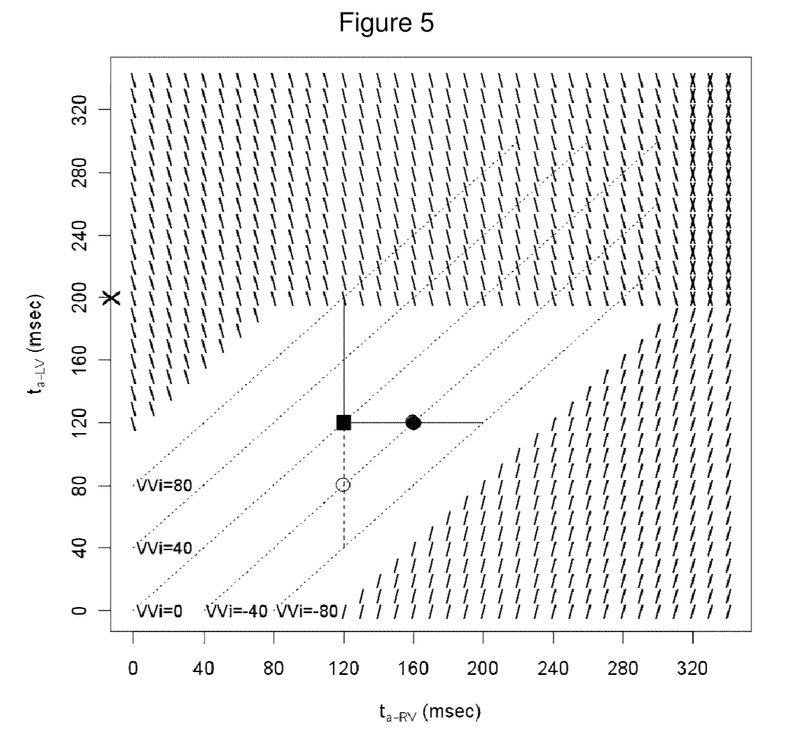

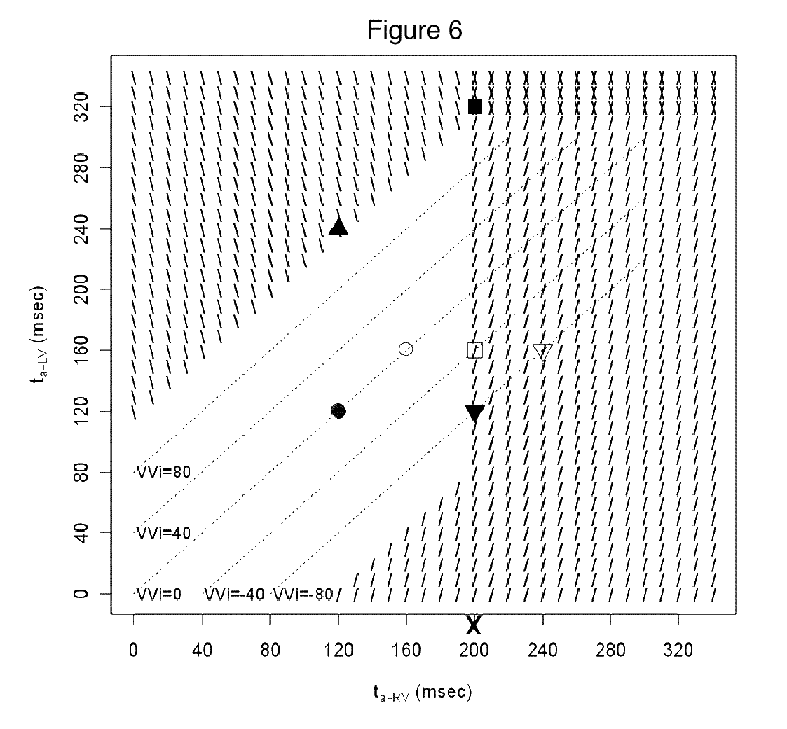

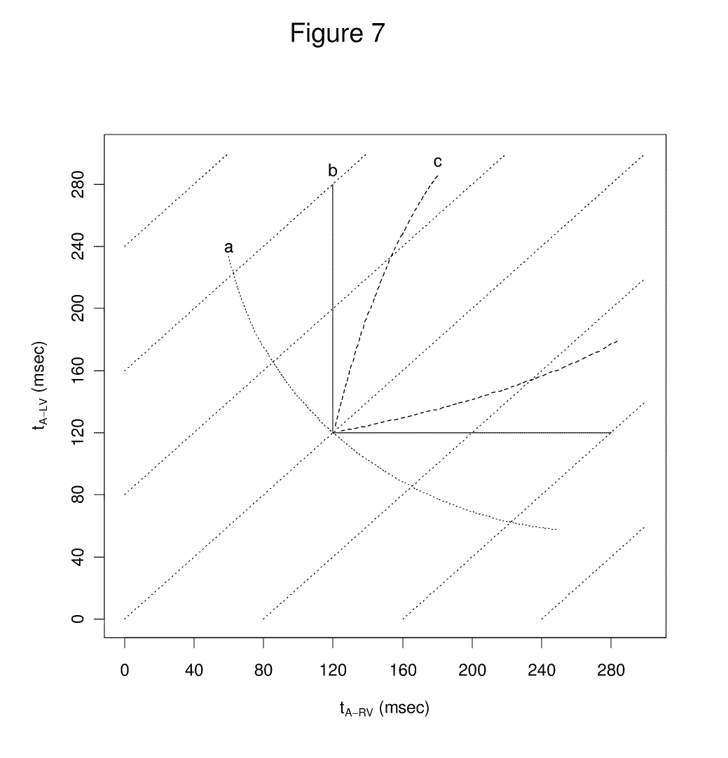

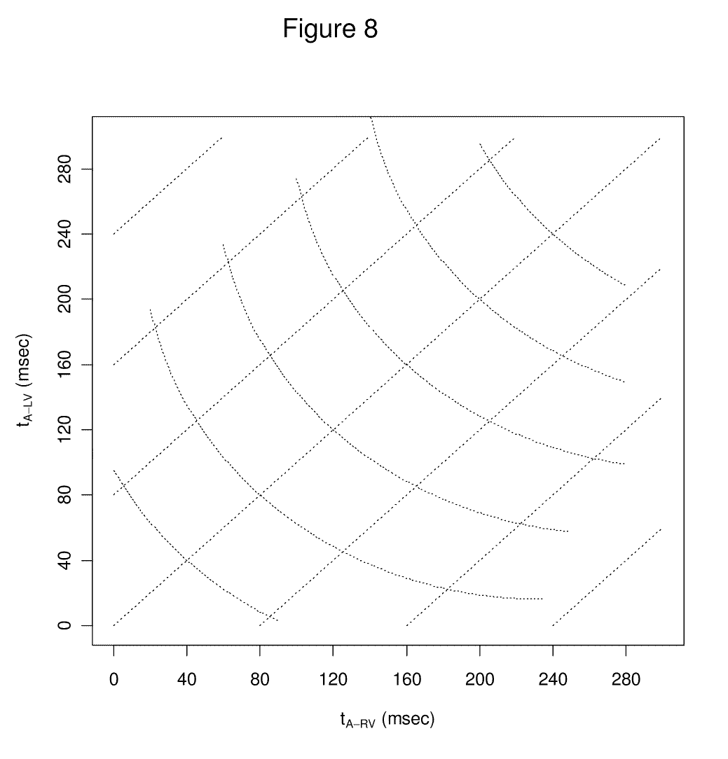

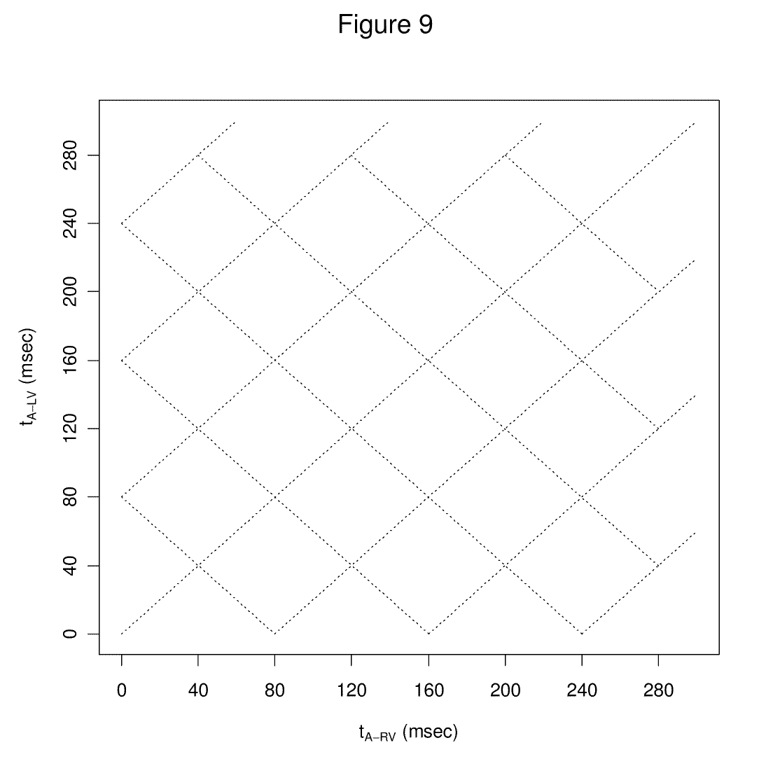

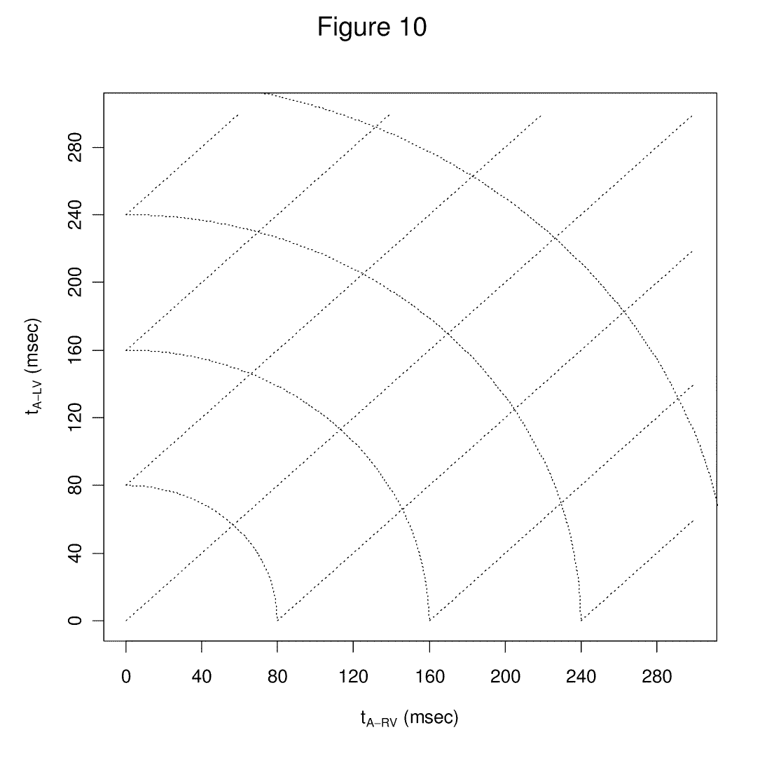

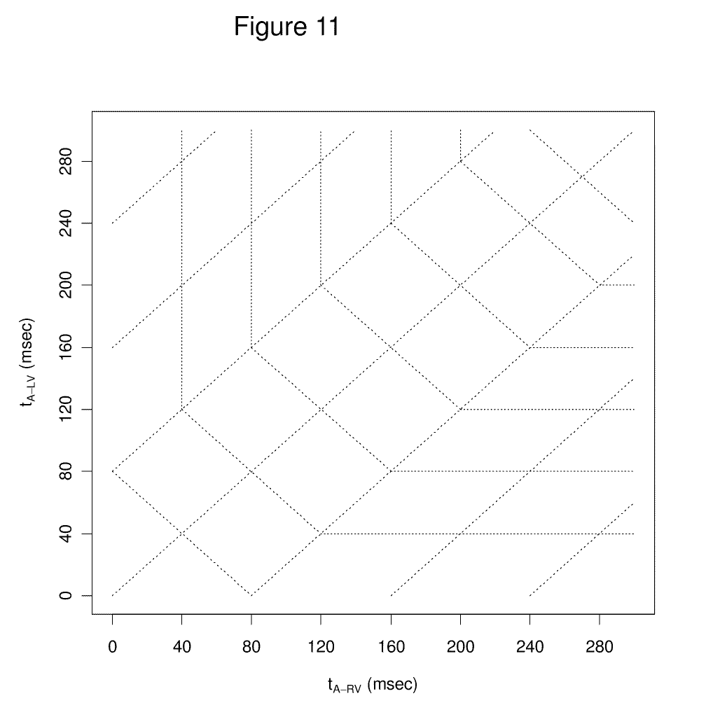

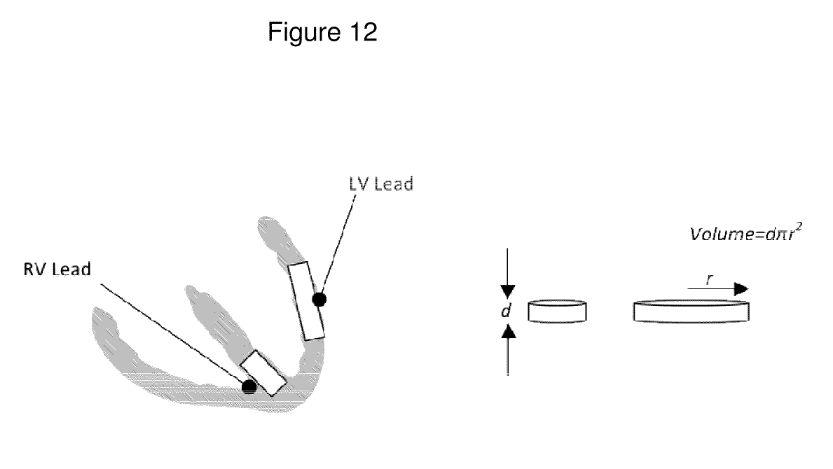

One aspect of the invention is centered on a new definition of and graphical representation of ventricular pace timing in which the times from atrial event (either sensed atrial activity or atrial pace) to LV pace and to RV pace are considered separately. These are denoted by ta_LV and ta_RV, respectively. Expressing pace timing in terms of ta_LV and ta_RV advantageously avoids ambiguity and facilitates changes in pace timing whereby LV preload and synchrony are independently adjusted, in contrast to conventional definitions of AVD and VVI in which changes in VVI result in changes in both synchrony and preload, despite holding the programmed AVD constant. As shown in FIG. 1, this representation allows a unique and unambiguous representation of ventricular timing events, including both pace timing and intrinsic conduction. It allows representation of VVI and preload isochrones, i.e., collections of ta_LV and ta_RV values that correspond to fixed VVIs and preload, respectively. The representation provides a convenient tool for the user to adjust pacing interval settings so that preload and synchrony are independently optimized. Furthermore the new representation allows one-click programming, in which the AVD and VVI (equivalently, the ta-RV and ta-LV) can be changed with a single touch of the screen at the location of the desired pacing intervals. In addition, the new representation allows lock-out conditions to be displayed and easily interpreted. Such lock-out conditions are due to dependencies among various programmable parameters and occur when certain parameter values conflict with others, and are thus not allowed. For example, wide VV intervals may conflict with certain pace refractory and tachycardia detection values.

Pace timing in biventricular pacemakers is generally expressed in terms of the AV delay and VV interval. While these terms are intuitively appealing, they are imprecise and are implemented differently by different manufacturers. For example, some manufacturers define the AV delay as the time to first ventricular pace, while others define it as the time to RV pace.

Working directly in terms of ta-LV and ta-RV, the time between atrial event and LV and RV paces, respectively, avoids this ambiguity. Combining the parameters to construct the ta-LV/ta-RV plane allows a graphical representation of pace timing as well as the timing of other ventricular events, such as onset of intrinsic conduction. In this representation each point in the plane corresponds to a unique RV and LV pace timing, and therefore a unique AV delay and VV interval. The atrial event can be either atrial sense or atrial pace; in practice one would use separate ta-LV/ta-RV planes for each, or superimpose the information on a single representation.

FIG. 5 illustrates a programmed AV delay of 120 msec with simultaneous biventricular pacing (solid square). Dotted lines represent the interventricular interval isochrones, which correspond to the collections of ta-LV and ta-RV values that yield fixed interventricular pacing intervals. Thus, as with the filled square, a pair of ta-LV and ta-RV that falls anywhere on the principal diagonal, labeled VVi=0, will correspond to simultaneous biventricular pacing. Holding ta-LV fixed at 120 and extending ta-RV to 160 corresponds to pacing at the point marked by the solid circle. This falls on the VVi=−40 isochrone, indicating a VV interval of 40 with LV preceding RV. Holding ta-RV fixed at 120 while ta-LV is shortened to 80 msec (open circle) similarly falls on the VVi=−40 isochrone, again corresponding to an interventricular pacing interval of 40 msec with LV preceding RV.

The open and solid circles represent the delivered pace timing for different manufacturers when identical programmed parameters are used, i.e., an AV delay of 120 msec and VV interval of 40 msec, LV first. For example, Medronic and St Jude Medical define the AV delay as the time to first ventricular pace, so with a programmed AV delay of 120, pacing the LV first requires holding ta-LV fixed at 120 while ta-RV is extended. In this case the point representing the ta-LV, ta-RV pair moves along the solid horizontal line. For these manufacturers, a programmed AV delay of 120 with RV paced first is implemented by holding ta-RV fixed at 120 while ta-LV is lengthened. This corresponds to moving along the vertical solid line.

In contrast, Boston Scientific defines the programmed AV delay as the time to RV pace, and has historically implemented negative VV intervals (LV before RV) but not positive ones. For its devices, programming a fixed AV delay of 120 while extending the VV interval (LV first) corresponds to moving along the dashed line in the figure. Representation of the programmed AV delay isochrones in the ta-LV/ta-RV plane clearly illustrates the divergent implementations by different manufactures. In addition, the abrupt 90 degree transition of the programmed AV delay isochrone as the early-paced ventricle changes from one side to the other suggests this implementation is driven by more by engineering considerations than physiology.

FIG. 5 also represents intrinsic RV and LV conduction for a patient with a right bundle branch block. Loss of ventricular capture is denoted with forward slash (‘/’) for the RV and backslash (‘\’) for the LV. The region of the plane that is free of either slash corresponds to pace-timing pairs that result in biventricular capture. The region that has both slashes corresponds to pace-timing that would not capture either chamber because intrinsic conduction has already occurred. Intrinsic conduction through the AV node results in a vertical (RV) or horizontal (LV) boundary since loss of capture in one chamber is independent of the pace-timing of the contralateral chamber. FIG. 5 illustrates intrinsic conduction through the AV node to the LV with a delay of 200 msec, demarcated on the ta-LV axes with an ‘x.’ In contrast, conduction from a contra-lateral pace results in a boundary that follows a VV isochrone, so that extending the interval of the paced chamber would delay conduction to the contralateral chamber by the same amount. In this example conduction time from a contralateral pace is illustrated using a delay of 120 msec, though in general it would not be symmetric. It should be noted that the shape of the boundaries may diverge from this idealized illustration due to changes in conduction velocity in various regions of the plane. Furthermore, the boundaries may shift in time due to changes in autonomic tone, circulating catacholamines, degree of ischemia, medication, and other factors.

FIG. 6 illustrates intrinsic conduction patterns in the setting of left bundle branch block (LBBB). Native conduction reaches the RV via the AV node 200 msec after the atrial event, indicated on the ta-RV axis with an ‘x.’ Simultaneous biventricular pacing at 160 msec is represented by the open circle on the VVi=0 isochrone. Holding ta-LV fixed at 160 while extending ta-RV to 240 (open triangle) would result in loss of RV capture due to intrinsic conduction. Since any pacing combination with ta-LV=160 and ta-RV>=200 results in the same electrical event (LV pace at 160 with intrinsic RV conduction), we map intervals that result in loss of capture to the boundary denoting the onset of intrinsic conduction, illustrated in the Figure with an open square. Thus, simultaneous biventricular pacing at 120 msec is represented by the filled circle on the VVi=0 isochrone, LV only pacing at 120 is located at the downward pointing solid triangle, RV only pacing at 120 is indicated by the upward pointing solid triangle, and fully intrinsic conduction (no pace capture in either ventricle) is indicated by the filled square.

Native conduction can be estimated automatically by the biventricular device and/or programmer by automatically recording the time to intrinsic activation for each point in the plane. Alternatively pacing can be attempted at each point in the ta-RV and ta-LV plane and an assessment can be made about whether the pacemaker successfully captured or not, e.g., by analyzing the evoked response.

In a further aspect of the invention, atrioventricular optimization is achieved by converting the programmed electrical atrioventricular delay (AVD) and interventricular interval (VVI) into right ventricular (RV) and left ventricular (LV) pace timing such that adjustments to VVI maintain a fixed preload, i.e., occur along a given mechanical AVD isochrone. Preload isochrones describe collections of intervals between atrial events and ventricular paces (i.e., ta_LV and ta_RV) that yield a given ventricular preload.

In another aspect of the invention, the preload rather than the nominal programmed AVD is held constant to ensure constant preload, while the VVI is optimized. The preload can be measured, for example, using Doppler echocardiography to measure the mitral filling velocity-time integral, or using a LV pressure catheter to measure end-diastolic pressure. Surrogates of preload can also be used such as total diastolic filling time, degree of mitral regurgitation, etc.

The goals of atrioventricular delay (AVD) optimization are to improve left ventricular (LV) filling, timing of contraction and to minimize mitral regurgitation (Gasparini et al., 2002); as a consequence, AVD optimization increases cardiac output. The goal of interventricular interval or delay (VVI) optimization is to reduce left ventricular dyssynchrony to improve systolic performance (Bax et al., 2005). In conventional pacemakers, the optimal atrioventricular delay is typically determined by setting interventricular delay (VVI)=0 millisecond (ms) and then varying AVD until the optimal AVD is identified. The so identified optimal AVD is then utilized, while varying interventricular delay to find the optimal interventricular interval (Zuber et al., 2008). Alternatively, in conventional pacemakers, the optimal interventricular interval is typically determined by setting AVD to a default value or arbitrarily determined value, while varying VVI until the optimal VVI is identified; then the so identified optimal VVI is used, while varying A VD to determine the optimal AVD (Zuber et al., 2008).

Preload isochrones are collections of ta_LV and ta_RV that yield a constant preload. Thus, in the plane defined by ta_LV and ta_RV, moving along a particular preload isochrone results in a changing VVI while preload is held fixed. Note that this will in general also be associated with changes in programmed electrical AVD, but that is acceptable because the programmed electrical AVD is an arbitrarily defined pacemaker parameter, and it is the preload, not the programmed AVD, that should be held fixed as the VVI is adjusted.

Optimization along preload isochrones improves cardiac function, which is critically important for the target patient population which, because of their severely compromised intrinsic cardiac function, faces a very high mortality rate with poor quality of life. Optimization along preload isochrones facilitates global optimization, i.e. identification of the overall best pacing combination. For example, in a practical setting, the clinician could determine the optimum optimal preload by adjusting the programmed AVD while holding VVI fixed (e.g., at 0 msec). Once the optimum preload was determined, the optimal VVI could be found by adjusting ta_LV and ta_RV such that the preload is held fixed while the VVI is varied (i.e., move along a preload isochrone). Thus, preload and synchrony are independently optimized in contrast to what is possible with current technology, namely, independent optimization of programmed VVI and programmed AVD. As noted above, this has the drawback of inextricably confounding changes in preload and synchrony.

In comparison with conventional pacemakers that require successive iterations during the course of the optimization process, optimization along preload isochrones is considerably more efficient, since it achieves global optimization in a single two-step process: 1) Optimization of preload 2) Optimization of synchrony. Importantly, operation along preload isochrones allows these to be done independently so there is not the inextricable confounding that is caused by conventional technology.

Time ambiguity is avoided by expressing timing in terms of intervals between an atrial event and left ventricular and right ventricular pace, for example ta-LV=interval between atrial pace or atrial sense and left ventricular pace; and ta-RV=interval between atrial pace or atrial sense and right ventricular pace. This pair of intervals defines a plane. As illustrated in FIG. 1, each point on the plane uniquely specifies a pace timing configuration. Electrical VVI isochrones are shown as dotted diagonal lines which represent the locus of points that correspond to a given interventricular interval (VVI). For example, simultaneous biventricular pacing, in which case VVI is zero, occurs at points along the diagonal that passes through the origin.

FIG. 1 also displays the preload isochrones for 40, 80, 120, 160, and 180 msec isochrones. These are the loci of LV and RV pacing timing combinations (i.e., pairs of ta_LV and ta_RV) that result in a given preload, specifically, the preload associated with the indicated programmed AVD (i.e., 40, 80, 120, etc) when VVI=0.

The particular preload isochrones shown in FIG. 1 are mechanical AVD isochrones, i.e., the collection of ta-RV, ta-LV that yield constant mechanical AVD. In contrast to the programmed AVD, which is the time from atrial event to ventricular pace, the mechanical AVD is the time from atrial event to the beginning of ventricular systole, marked, for example, by the closure of the mitral valve or the that time at which dP/dt exceeds 10% of its maximum. Mechanical AVD isochrones can be estimated theoretically or empirically, as described below. Effects that tend to increase preload as the VVI is widened with a fixed programmed AVD will, similar to mechanical AVD isochrones, form an angle with the principal diagonal that is greater than 90 degrees. Examples of other mechanisms that have this effect include reduced synchrony, which in turn reduces contractility and the amount of blood ejected with each heart beat, thus resulting in greater residual ventricular volume at the end of systole. Isochrones refer to collections of parameter values that result in a particular value of a property of interest, such as ventricular preload. As illustrated here in the preferred embodiment they are represented as curves in the ta-LV,ta-RV plane. This does not exclude the use of other representations of isochrones, such as a 3 dimensional surface over the two-dimensional plane, which, as with the isochrones illustrated here, would also represent collections of values of the independent variables that result in particular values of the property of interest.

In contrast, mechanisms that tend to decrease preload as the VVI is widened with a fixed programmed AVD will form an angle with the principal diagonal that is less than 90 degrees, as illustrated in FIG. 7 curve ‘c’. Examples include the increased isovolumic contraction and relaxation times which result in worsening synchrony and hence shorter diastolic filling time and increased presystolic mitral regurgitation. Thus, in FIG. 7, the dotted line (labeled ‘a’) is a single isochrone that results when the preload decreases as the programmed AV delay is held fixed and the VV interval is widened. It shows the collection of ta-RV, ta-LV that result in the same preload as AVD=120, VVi=0. The solid line of FIG. 7, labeled ‘b’, results when no change in preload occurs as the VVi is changed and the programmed AVD is held fixed. Note that this is the implicit though seldom recognized assumption in conventional pacing interval optimization, in which the VVi is changed and the programmed AVD is held fixed. The dashed line in FIG. 7, labeled ‘c’, forms an angle with the principal diagonal that is less than 90 degrees. This corresponds to a preload that decreases as the VVi is widened and the programmed AVD is held fixed.

FIG. 8 shows individual isochrones from a family of isochrones that are concave-up. FIG. 9 shows individual isochrones from a family that is orthogonal to the VVi isochrones. In this case preload remains fixed if the average of ta-RV and ta-LV is constant. FIG. 10 illustrates an example of concave-down isochrones. FIG. 11 shows piece-wise linear isochrones, in which for small VVi preload is constant for constant average time to biventricular pace (i.e., average of ta-RV and ta-LV), while for larger VVi preload is determined entirely by the time to first ventricular pace. FIGS. 8-11 all illustrate isochrone families in which preload increases as the VVi is widened with a fixed programmed AVD.

It should be noted that preload isochrones can be asymmetric about the principal diagonal. Furthermore, a few individual members from each family are illustrated for convenience. There is a continuum of curves in each family, each of which corresponds to a unique value of programmed AVD with VVi=0. In addition, isochrones may take on different appearances in different regions of the ta-RV, ta-LV plane, e.g., be concave up in one region and concave down in another.

The ta-RV, ta-LV plane can display isochrones that correspond to specific effects, or composite isochrones that represents the overall pre-systolic loading seen by the ventricle. The complete or composite preload isochrone might have the concave up appearance shown in FIG. 1, the concave down appearance shown in FIG. 10, or the linear appearance shown in FIG. 9, or a combination, such as that shown in FIG. 11. Note that the linear appearance seen in FIG. 9, in which the preload isochrones are orthogonal to the VVI isochrones, correspond to the average of ta-RV and ta-LV.

Theoretical prediction of such AVD isochrones is possible. A simple model of cardiac function predicts that for a given mechanical AVD isochrone, ta_LV and ta_RV are related by t_{a-LV}=τ+\sqrt{k−(τ−t_{a-RV})²} where tau is the mechanical AVD, i.e., the time between the atrial event and the end of ventricular filling, and k is a constant that reflects biophysical properties of the left ventricle, including wavefront conduction velocity, the critical mass of myocardium that must be excited to close the mitral valve and terminate diastolic filling, the thickness of the myocardium, and the density of the myocardium. To empirically measure k, one could measure the time from atrial event to mitral valve closing while simultaneously pacing both ventricles using a programmed AVD of 120 msec, giving k=2(τ−120)². Alternatively, mechanical AVD isochrones can be empirically determined for populations of patients. Still another alternative is to empirically determine mechanical AVD isochrones for each individual patient, for example, my measuring time to mitral valve closure or time to increase in LV pressure at a variety of programmed interval settings.

Rather than representing mechanical AVD isochrones or other specific isochrones such as diastolic filling time, the more general preload isochrones can be represented. These can be empirically determined for the individual patient or for populations of patients by recording, e.g., the LV end-diastolic pressure over the ta-RV, ta-LV plane. Similarly, they can be estimated by measuring the mitral inflow velocity-time integral or ventricular wall stress. Alternatively, the preload isochrones can be estimated from theoretical considerations. Specifically, as illustrated in FIG. 12, with respect to the left ventricle, the RV and LV leads are fairly symmetrically placed, with the LV lead typically activating the LV free wall and the RV lead activating the distal septum and apex. Thus compared to VVI=0 (with ta-RV=ta-LV=AVD₀=constant), for symmetric changes in ta-RV and ta-LV such that ta-RV=AVD₀+delta and ta-LV=AVD₀−delta, by symmetry we would expect the loading conditions on the LV to remain unchanged. In other words, the preload remains constant when the average of ta-RV and ta-LV is constant. This model thus predicts the orthogonal preload isochrones illustrated in FIG. 9 when delta is small. However, when delta becomes large enough the depolarization wavefront from the early-paced lead will have enough time to sweep across the LV before the contralateral lead is placed. At this point the timing of the contralateral pace becomes irrelevant so that the isochrones become parallel to the axes, as illustrated in FIG. 11. The inflection point of the isochrone, i.e., the point at which it changes from orthogonal to the VVI isochrone to parallel to the axes, can be estimated empirically by electrophysiology studies in which propagation velocity is recorded or arrival time at various points in the LV are noted.

The method of programming or parameter conversion that preserves preload can use either theoretical transformation equations or empiric data. For example, the optimum preload can be determined by optimizing cardiac function as the VVI is held fixed (eg, at 0 msec) and the programmed AVD is adjusted. For this step, adjustments are made along a VVI isochrone. Once the optimum preload corresponding to the optimum programmed AVD with VVI=0 is identified, synchrony can be optimized by adjusting the VVI (and possibly programmed AVD) such that preload is held fixed. For this step, adjustments are made along a preload isochrone.

In one embodiment, the programmer would display a graphical representation of the timing information (similar to FIG. 1). It would provide a preload isochrone that passes through or near to the just-determined optimum programmed AVD. By visual inspection, the user could then select new pacing configurations that fell along the same preload isochrone.

In an alternative embodiment, the user could specify a new test VVI and the programmer could calculate the corresponding ta_LV and ta_RV pairs that yielded the specified VVI while maintaining the previously determined preload. For example, using the model referred to above the programmer could solve the equation so that the difference between ta_LV and ta_RV was equal to the selected VVI.

In still another alternative embodiment a modified theoretical model could be used to provide the mathematical formulas. Such a model might take into account patient-specific characteristics such as ventricular anatomy, size and location of scars from previous myocardial infarctions, and lead positions.

In yet another embodiment the programmer could use empirical data to obtain the appropriate values for ta_LV and ta_RV that yield the specified VVI while maintaining a fixed ventricular preload. Such empirical data could be obtained from populations of patients or from the individual patient whose pacemaker is currently being programmed.

In another embodiment optimum pacing parameters estimated from electrogram conduction times can be displayed, e.g., based on theoretical considerations or population-derived associations (U.S. Pat. No. 7,643,878; Stein et al., 2009).

Pace-timing information can be displayed in terms of ta-RV and ta-LV, with or without presentation of ta-RV, ta-LV plane.

In a further aspect of the invention, nominal electrical programmed AVD and VVI can be converted to pace-timing based on fixed mechanical AVD isochrones.

In yet another aspect of the invention, a specified LV or RV pace timing and desired VVI can be converted to preload isochrone and contralateral pace timing, which may be useful for clinicians who are more familiar with conventional definitions. The transformations are manufacturer specific. For example, for Medtronic and St Jude devices, the programmed AVD is equal to the minimum of ta_RV and ta_LV, and the programmed VVI is equal to the magnitude of the difference between ta_RV and ta_LV.

Various formulae are used to convert nominal pacing intervals to ventricular pace timing on a given preload isochrone and to obtain preload isochrones. In addition, provision is made for generating pace timing when preload is determined empirically including lookup table and interpolation.

Implantable cardiac stimulation devices are usually configured to be used in conjunction with an external programmer which allows the physician enter certain parameters to control the operation of the device. For instance, the physician may specify the sensitivity with which the pacemaker or ICD senses electrical signals within the heart and also specify the amount of electrical energy to be employed in pacing pulses or defibrillation shocks. Another common control parameter is pacing rate and pacing mode which determines which chambers of the heart are paced. In addition, the programmer provides a means to download data that has been stored by the pacemaker or ICD, for example, the history of heart rates and rhythms.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are herein described.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### Experimental Procedures

The following model is put forth so as to provide those of ordinary skill in the art with a complete disclosure and description of how to make and use the present invention; it is not intended to limit the scope of what the inventors regard as their invention.

We express pace timing in terms of the time from atrial event (sensed or paced) to RV pace (tA-RV) and to LV pace (tA-LV); these two parameters define a plane in which each point uniquely corresponds to a pair (tA-RV,tA-LV). A preload isochrone is defined as the collection of (tA-RV,tA-LV) pairs that correspond to a given preload. Preload is determined by a number of distinct mechanisms including mechanical AVD, pre-systolic mitral regurgitation, and diastolic filling time. It may be desirable to represent an individual mechanism such as mechanical AVD or approximate the overall preload status. A mechanical AVD isochrone is defined as the collection of (tA-RV,tA-LV) pairs that corresponds to a given mechanical AVD.

We define the mechanical AV delay as the time from atrial sense or pace to the end of ventricular filling. The dependence of mechanical AVD on A-LV and A-RV pacing intervals was modeled using the following 4 assumptions: 1) Ventricular filling ends when a critical mass of LV myocardium is activated; 2) The three-dimensional structure of the LV is equivalent to a two dimensional plane from the perspective of wavefront propagation; 3) Propagations proceeds radially from the point of activation with a constant, uniform velocity; 4) LV and RV paces both contribute symmetrically to LV contraction.

A critical mass of myocardium mc determines the end of the mechanical A V delay and has contributions from both the RV and LV paces:

m_{c}dpτr_{R}²+dpτr_{R}², (1)

where d is the thickness of the myocardium, ρ is its density, and r is the radius of wavefront propagation in LV myocardium by RV and LV paces. Since the wavefront propagates with uniform conduction velocity v, at the end of the mechanical AV delay the radii of the volumes associated with RV and LV paces are

r_{L}=ν(τ−t_{a-LV}) and r_{R}=ν(τ−t_{a-RV}).

τ is the mechanical AV delay.

Substituting into Eq. 1 gives

m_{c}=dpπν²{(τ−t_{a-RV})²+(τ−t_{a-LV})²}. (2)

Rearranging and applying the quadratic formula gives

(τ−t_{a-RV})²+(τ−t_{a-LV})²=m_{c}/dpπν²≡k (3)

t_{a-LV}=τ+\sqrt{k−(τ−t_{a-RV})²}. (4)

We can solve for the mechanical AV delay τ by expanding Eq. 2 and applying the quadratic formula, which yields

$$τ = \frac{1}{2} \{ \begin{matrix}( {t}_{a - LV} + {t}_{a - LV} ) + \\ \sqrt{2 k - {( {t}_{a - RV} - {t}_{a - LV} )}^{2}}\end{matrix} \} , if \vert {t}_{a - LV} - {t}_{a - LV} \vert \leq \sqrt{k} \\ τ = min ( {t}_{a - LV} , {t}_{a - LV} ) + \sqrt{k} , if \vert {t}_{a - LV} - {t}_{a - LV} \vert > \sqrt{k} . \qquad ( 5 )$$

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Abraham W T et al. (2002), “Cardiac resynchronization in chronic heart failure.” N Engl J Med 346, pp. 1845-1853.
Agler D A et al. (2007), “Cardiac resynchronization therapy and the emerging role of echocardiography (part 2): the comprehensive examination.” J Am Soc Echocardiogr 20, pp. 76-90.
Auricchio A et al. (2002), “Cardiac resynchronization therapy restores optimal atrioventricular mechanical timing in heart failure patients with ventricular conduction delay.” J Am Coll Cardiol 39, pp. 1163-1169.
Bax J J et al. (2005), “Cardiac resynchronization therapy. Part 2—Issues during and after device implantation and unresolved questions.” J Am Coll Cardiol 46, pp. 2168-2182.
Bertini M et al. (2008), “Interventricular delay interval optimization in cardiac resynchronization therapy guided by echocardiography versus guided by electrocardiographic QRS interval width.” Am J Cardiol 102, pp. 1373-1377.
Boriani G et al. (2006), “Randomized comparison of simultaneous biventricular stimulation versus optimized interventricular delay in cardiac resynchronization therapy: The Resynchronization for the HemodYnamic Treatment for Heart Failure Management II implantable cardioverter defibrillator (RHYTHM II ICD) study.” Am Heart J 151, pp. 1050-1058.
Braun M U et al. (2005), “Impedance cardiography as a noninvasive technique for atrioventricular interval optimization in cardiac resynchronization therapy.” J Interv Card Electrophysiol 13, pp. 223-229.
Burri H et al. (2006), “Optimization of device programming for cardiac resynchronization therapy.” Pacing Clin Electrophysiol 29, pp. 1416-1425.
Burri H et al. (2005), “Optimizing sequential biventricular pacing using radionuclide ventriculography.” Heart Rhythm 2, pp. 960-965.
Cazeau S et, al (2001), “Effects of multisite biventricular pacing in patients with heart failure and intraventricular conduction delay.” N Engl J Med 344, pp. 873-880.
Chung E S et al. (2008), “Results of the Predictors of Response to CRT (PROSPECT) trial.” Circulation 117, pp. 2608-2616.
Gasparini M et al. (2002), “Optimization of cardiac resynchronization therapy: technical aspects.” Eur Heart J 4, pp. D82-D87.
Heinroth K M et al. (2007), “Impedance cardiography: a useful and reliable tool in optimization of cardiac resynchronization devices.” Europace 9, pp. 744-750.
Jansen A H et al. (2006), “Correlation of echo-Doppler optimization of atrioventricular delay in cardiac resynchronization therapy with invasive hemodynamics in patients with heart failure secondary to ischemic or idiopathic dilated cardiomyopathy.” Am J Cardiol 97, pp. 552-557.
Morales M A et al. (2006), “Atrioventricular delay optimization by doppler-derived left ventricular dP/dt improves 6-month outcome of resynchronized patients.” Pacing Clin Electrophysiol 29, pp. 564-568.
Stein K M et al. (2009), “Smartdelay determined av optimization: A comparison of av delay methods used in cardiac resynchronization therapy (smart-av): Rationale and design.” Pacing Clin Electrophysiol. 2009, pp 1-10.
Tse H F et al. (2003), “Impedance cardiography for atrioventricular interval optimization during permanent left ventricular pacing.” Pacing Clin Electrophysiol 26, pp. 189-191.
Turcott R G & Pavek T J. (2008), “Hemodynamic sensing using subcutaneous photoplethysmography.” Am J Physiol Heart Circ Physiol 295, pp. H2560-H2572.
van Gelder B M et al. (2008), “The Optimized V-V Interval Determined by Interventricular Conduction Times Versus Invasive Measurement by LVdP/dt(MAX).” J Cardiovasc Electrophysiol 19, pp. 939-944.
Vidal B et al. (2007), “Electrocardiographic optimization of interventricular delay in cardiac resynchronization therapy: a simple method to optimize the device.” J Cardiovasc Electrophysiol 18, pp. 1252-1257.
Zuber M et al. (2008), “Comparison of different approaches for optimization of atrioventricular and interventricular delay in biventricular pacing.” Europace 10, pp. 367-373.

What is claimed is:

1. A method for specifying ventricular pace timing, the method comprising an evaluation of ventricular loading conditions whereby the evaluation of ventricular loading conditions is based on an evaluation of preload isochrones describing a collection of timing events
from atrial event to left ventricular pace (‘tA-LV’) and
from atrial event to right ventricular pace (‘tA-RV’).

2. The method of claim 1 wherein the atrial event is sensed atrial activity.

3. The method of claim 1 wherein the atrial event is atrial pace.

4. A method for graphically representing ventricular pacing and timing events, the method comprising displaying on a first axis timing information for a first parameter and on a second axis timing information for a second parameter.

5. The method of claim 4, wherein said first parameter is a programmed atrioventricular delay and said second parameter is a programmed interventricular interval.

6. The method of claim 4, wherein said first parameter is tA-RV and said second parameter is tA-LV.

7. The method of claim 4, wherein one or more intrinsic electrical events are displayed.

8. The method of claim 4, wherein selectable programmable settings are displayed.

9. The method of claim 4, wherein currently programmed and previously programmed parameter settings are displayed.

10. The method of claim 4, wherein isochrones are displayed.

11. The method of claim 10, wherein the isochrones are interventricular interval (VVi) isochrones.

12. The method of claim 10, wherein the isochrones are atrioventricular delay (AVD) isochrones.

13. The method of claim 4, wherein the display of isochrones can be changed with a single user input (‘one-click programming’).

14. The method of claim 7, wherein the one or more intrinsic electrical events are intrinsic conduction to a ventricle.

15. A method for processing interval values for use in delivering cardiac pacing therapy to a heart of a patient in which an implantable cardiac stimulation device is implanted, the method comprising: determining a nominal electrical preload value; determining a nominal electrical interventricular delay; converting said nominal electrical preload value and said nominal electrical interventricular delay (VVI) into ventricular pace timing based on preload isochrones.

16. The method of claim 15, wherein the interval values are interventricular interval values.

17. The method of claim 15, wherein the interval values are preload values.

18. A system for graphically representing ventricular pacing and timing events, the system comprising means for displaying on a first axis timing information for a first parameter and on a second axis timing information for a second parameter.

19. The system of claim 18, wherein said first parameter is a programmed atrioventricular delay and said second parameter is a programmed interventricular interval.

20. The system of claim 18, wherein said first parameter is tA-RV and said second parameter is tA-LV.

21. The system of claim 18, wherein one or more intrinsic electrical events are displayed.

22. The system of claim 18, wherein selectable programmable settings are displayed.

23. The system of claim 18, wherein currently programmed and previously programmed parameter settings are displayed.

24. The system of claim 18, wherein isochrones are displayed.

25. The system of claim 24, wherein the isochrones are interventricular interval (VVi) isochrones.

26. The method of claim 24, wherein the isochrones are atrioventricular delay (AVD) isochrones.

27. The system of claim 18, wherein the display of isochrones can be changed with a single user input (‘one-click programming’).

28. A method for adjusting programmable parameter values of a pacemaker wherein multiple parameter values are specified with a single user input.

29. The method of claim 28 wherein combinations of selectable parameter values are simultaneously displayed.

30. A system for adjusting programmable parameter values of a pacemaker wherein multiple parameter values are specified with a single user input.

31. The system of claim 30 wherein combinations of selectable parameter values are simultaneously displayed.
